(19) United States
(12) Patent Application Publication
(10) Pub. No.: US 2026/0241119 A1
(43) Pub. Date: Aug. 20, 2026

(54) METERED DOSE INHALERS AND SOLUTIONS INCLUDING CANNABINOIDS IN HFA-152A

(71) Applicant: Kindeva Drug Delivery L.P., Woodbury, MN (US)

(72) Inventors: Philip Cocks, West Bridgford (GB); Sarah Wrigglesworth, Loughborough (GB)

(21) Appl. No.: 19/161,043

(22) PCT Filed: Mar. 1, 2024
(86) PCT No.: PCT/US2024/018030

§ 371 Date: Aug. 29, 2025

Related U.S. Application Data
(60) Provisional application No. 63/449,438, filed on Mar. 2, 2023.

(51) Int. Cl. A61M 15/00; A61K 9/00; A61K 9/08; A61K 31/00; A61K 47/10; A61M 11/02; A61M 39/22

(52) U.S. Cl. CPC A61M 15/009; A61K 9/008; A61K 9/08; A61K 31/658; A61K 47/10; A61M 11/02; A61M 39/22; A61M 2039/226

(57) ABSTRACT

Various embodiments of a metered dose inhaler are disclosed. The inhaler includes a metering valve, a canister, and an actuator having an actuator nozzle. The canister includes a formulation having a propellant including HFA-152a, at least 1% of ethanol by weight, and one or more cannabinoids, wherein the one or more cannabinoids is dissolved in the formulation to form a solution. In one or more embodiments, the cannabinoid includes tetrahydrocannabinol (THC) and/or cannabidiol (CBD).

### CROSS-REFERENCE TO RELATED APPLICATION

This application claims the benefit of U.S. Provisional Patent Application Ser. No. 63/449,438, filed on Mar. 2, 2023, which is incorporated by reference herein in its entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. Metered dose inhalers are familiar to many patients who suffer from asthma or chronic obstructive pulmonary disease (COPD). Metered dose inhaler devices can include an aluminum canister, sealed with a metering valve, that contains a medicament formulation. Generally, a typical current medicament formulation includes one or more medicinal compounds present in a liquefied hydrofluoroalkane (HFA) propellant.

One such type of medicament are cannabinoids. Cannabinoids are the main active component of Cannabis and are known to have various neurological effects. A cannabinoid may be combined with a liquified propellant to prepare a formulation to be delivered using a metered dose inhaler.

Historically, the propellants in most metered dose inhalers have been chlorofluorocarbons (CFCs). However, environmental concerns during the 1990s led to the replacement of CFCs with hydrofluoroalkanes (HFAs) as the most commonly used propellant in metered dose inhalers. Although HFAs do not cause ozone depletion, they do have a stated high global warming potential (GWP), which is a measurement of the future radiative effect of an emission of a substance relative to that of the same amount of carbon dioxide (CO₂). The two HFA propellants most commonly used in pMDIs are HFA-134a, also called HFC-134a, R-134a, or norflurane (CF₃CH₂F, 1,1,1,2-tetrafluoroethane) and HFA-227, also called HFC-227, FM-200, or apaflurane (CF₃CHFCHF₃, 1,1,1,2,3,3,3-heptafluoropropane) having stated 100-year GWP values of 1300 to 1430 and 3220 to 3350, respectively.

Various other propellants have been proposed over the years. Among them, hydrofluoroolefins (HFOs) and carbon dioxide (CO₂) have been mentioned as potential propellants for metered dose inhalers, but still a need exists for metered dose inhaler products using either as a propellant.

### SUMMARY

It has now been found that despite HFA-152a's differences from other pMDI propellants, a practical pMDI can be made using HFA-152a. One advantage of such pMDIs is HFA-152a's low stated GWP.

In one embodiment, a pMDI (also referred to herein as an MDI or a metered dose inhaler) is provided that includes: a metering valve; a canister; and an actuator that includes an actuator nozzle; wherein the canister includes a formulation (i.e., a composition), the formulation including a propellant including HFA-152a, at least 1% of ethanol by weight, and one or more cannabinoids; and wherein the one or more cannabinoids is dissolved in the formulation to form a solution. In certain embodiments, the one or more cannabinoids includes tetrahydrocannabinol (THC), cannabidiol (CBD), or a combination thereof.

In one embodiment, a pMDI is provided that includes: a metering valve; a canister; and an actuator that includes an actuator nozzle; wherein the canister includes a formulation, the formulation including a propellant including HFA-152a, at least 1% of ethanol by weight, and CBD; and wherein the CBD is dissolved in the formulation to form a solution. In one embodiment, a pMDI is provided that includes: a metering valve; a canister; and an actuator that includes an actuator nozzle; wherein the canister includes a formulation, the formulation including a propellant including HFA-152a, at least 1% of ethanol by weight, and THC; and wherein the THC is dissolved in the formulation to form a solution.

Herein, “dissolved in the formulation” or “dissolved in the composition” means that the recited components (e.g., cannabinoids) are dissolved in the propellant, or dissolved in the propellant and other components such as a cosolvent, to form a solution.

Herein, the term “comprises” and variations thereof do not have a limiting meaning where these terms appear in the description and claims. Such terms will be understood to imply the inclusion of a stated step or element, or group of steps or elements, but not the exclusion of any other step or element, or group of steps or elements. The phrase “consisting of” means including, and limited to, whatever follows the phrase “consisting of” Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may, or may not, be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

Throughout this disclosure, singular forms such as “a,” “an,” and “the” are often used for convenience; singular forms are meant to include the plural unless the singular alone is explicitly specified or is clearly indicated by the context.

As used herein, the term “or” is generally employed in its usual sense including “and/or” unless the content clearly dictates otherwise.

The term “and/or” means one or all of the listed elements or a combination of any two or more of the listed elements.

The phrase “ambient conditions” as used herein, refers to an environment of room temperature (approximately 20° C. to 25° C.) and 30% to 60% relative humidity.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50). Herein, “at least” a number (e.g., at least 50) includes the number (e.g., 50). Herein, “no more than” a number (e.g., no more than 50) includes the number (e.g., 50). Herein a number “or greater (e.g., 50 or greater) includes the number (e.g., 50).

Numerical ranges, for example “between x and y” or “from x to y”, include the endpoint values of x and y. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and metered dose inhalers such as pressurized metered dose inhalers (pMDIs), and methods of this disclosure; this terminology is not used to mean that these features are present, much less common, in the prior art. Unless otherwise specified, only the Background section of this Application refers to the prior art.

Reference throughout this specification to “one embodiment,” “an embodiment,” “certain embodiments,” “one or more embodiments,” “one aspect,” “an aspect,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

The above summary of the present disclosure is not intended to describe each disclosed embodiment or every implementation of the present disclosure. The description that follows more particularly exemplifies illustrative embodiments. In several places throughout the disclosure, guidance is provided through lists of examples, which examples may be used in various combinations. In each instance, the recited list serves only as a representative group and should not be interpreted as an exclusive or exhaustive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

The complete disclosure of all patents, patent applications, publications, and electronically available material cited herein are incorporated by reference in their entirety. In the event that any inconsistency exists between the present disclosure and the disclosure(s) of any document incorporated herein by reference, the present disclosure shall govern. The detailed description and examples herein have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### BRIEF DESCRIPTION OF THE DRAWINGS

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

### DETAILED DESCRIPTION

The formulations described herein include one or more cannabinoids. Cannabinoids are psychoactive compounds and are the main psychoactive component of Cannabis. The medicinal properties of cannabinoids have been known for many years including their use for treating or alleviating chronic pain, seizures, arthritis, nausea, neurodegenerative diseases, such as multiple sclerosis, cancer and HIV. They may also be effective as bronchodilators in the treatment of asthma and COPD. However, alongside their potential benefits are the less desirable effects, including the psychotropic effects and the risk of diseases such as cancer if the cannabinoids are inhaled by smoking.

It is known to deliver cannabinoids using inhaler devices, including metered dose inhalers, such as pressurized metered dose inhalers (pMDIs), in which the cannabinoids are delivered using a propellant. The cannabinoid is dissolved in a liquefied propellant and optional cosolvent and stored in a pressurized container, such as a pMDI canister. The container is then coupled to a suitable delivery device which typically includes a mouthpiece, a nozzle, and a valve assembly. Actuation of the valve assembly releases a dose of the cannabinoid/propellant mixture from the container that is then dispensed from the nozzle into the mouthpiece where it can be inhaled.

The term “cannabinoid” as used herein encompasses naturally occurring as well as synthetic and semi-synthetic cannabinoids. Cannabinoids may naturally exist in plants in the family Cannabaceae and extracts derived therefrom, such as hemp oil. Cannabaceae plants that may naturally produce cannabinoids include Cannabis indica, Cannabis sativa, and Cannabis ruderalis. Cannabinoids may be derived from wild-type Cannabaceae plants or genetically modified variants thereof, such as those generated from genetic crosses, self-crosses, or hybridization. Variants may include varieties characterized by chemical composition that naturally contain different amounts of the individual cannabinoids, such as Cannabis chemovars, or Cannabis sativa subspecies indica including the variants var. indica and var. kafiristanica.

Suitable cannabinoids include phytocannabinoids, which can be isolated from plants to produce highly purified extract or can be reproduced synthetically, e.g., tetrahydrocannabinols (THC), cannabidiol (CBD), cannabigerols (CBG), cannabichromenes (CBC), cannabinol (CBN), cannabinodiol (CBDL), cannabicyclol (CBL), cannabielsoin (CBE), cannabidivarin (CBDV), tetrahydrocannabivarin (THCV), cannabitriol (CBT), and cannabinol (CBN). Highly purified cannabinoid extracts are also included and defined as cannabinoids that have been extracted from the Cannabis plant and purified (to the extent that other cannabinoids and non-cannabinoid components that are coextracted with the target cannabinoids have been substantially removed). Highly purified cannabinoid extracts can include at least 90%, at least 91%, at least 92%, at least 93%, at least 94%, at least 95%, at least 96%, at least 97%, at least 98%, or at least 99% of cannabinoid. Suitable synthetic cannabinoids are compounds that have a cannabinoid or cannabinoid-like structure and are manufactured using chemical means rather than extracted from plants. Synthetic cannabinoids include cannabinoids structurally related to tetrahydrocannabinol (THC), cannabimimetics, and eicosanoids. More particular examples of synthetic cannabinoids for use in the present disclosure include nabilone, rimonabant, cannabicyclohexanol, JHW-018, JWH-073, and HU-210.

Two particular cannabinoids of interest are tetrahydrocannabinol and cannabidiol. Tetrahydrocannabinol (THC, C₂₁H₃₀O₂) refers to two isomers, Δ-8-THC ((6aR, 10aR)-6,6,9-trimethyl-3-pentyl-6a,7,10,10a-tetrahydrobenzo[c]chromen-1-ol) and Δ-9-THC ((6aR,10aR)-6,6,9-trimethyl-3-pentyl-6a,7,8,10a-tetrahydro-6H-benzo[c]chromen-1-ol). The formulations described as including THC herein may include Δ-9-THC and/or Δ-8-THC. THC is typically known for its psychoactive effects, but is also used in the treatment of pain, chronic diseases such as multiple sclerosis, and as an appetite stimulant.

Cannabidiol (CBD, C₂₁H₃₀O₂), 2-[(1R,6R)-6-Isopropenyl-3-methylcyclohex-2-en-1-yl]-5-pentylbenzene-1,3-diol, is another cannabinoid of interest. While there is limited evidence for the clinical efficacy of CBD, there is nonetheless interest in its use in managing neurological disorders. Unlike THC, CBD does not typically have psychoactive effects, and is thus generally less governmentally regulated.

Whole plant extract (WPE), the starting material of CBD and THC drug substances is the extract from the dried, ground and decarboxylated Cannabis flowers which is prepared with super critical carbon dioxide to yield WPE used to manufacture the CBD and THC drug substances. The materials and solvents used in the manufacturing processes of the drug substances are commonly used materials suitable for their intended purpose, purchased from reputable and controlled suppliers in compliance with predetermined quality standards.

The cannabinoids of the present disclosure may be prepared using any suitable method, such as pharmaceutical grade, commercial-scale extraction methods. As used herein “pharmaceutical grade” indicates compliance with guidelines defined by the International Council for Harmonization (ICH) of Technical Requirements for Pharmaceuticals for Human Use, including for example ICH, “Q3C (R7) guideline” for residual solvents which are allowed in low levels as impurities in pharmaceutical products or “Q6A guideline” for Dronabinol. In the United States Food and Drug Administration (FDA), relevant test methods include USP<1111>, USP<921>, USP<467>, USP<61>, USP<62> and USP<281>.

The extraction method may be from any “plant material” as well as exudates. “Plant material” as used herein includes plants, plant parts (e.g., bark, leaves, stems, roots, flowers, fruit, seeds), herbal Cannabis, dried Cannabis biomass, or Cannabis flowers. The plant material may be decarboxylated Cannabis plant material, used herein to refer to Cannabis plant material which has been subject to a decarboxylation step. Exudates include material falling within the definition of “botanical raw material” in the Guidance for Industry Botanical Drug Products Draft Guidance, August 2000, US Department of Health and Human Services, Food and Drug Administration Centre for Drug Evaluation and Research.

The extraction method may involve single or multiple-step extraction and chromatographic purification and may include the methods that have been disclosed in U.S. Pat. No. 7,344,736 (Whittle et al.) and PCT Application Publication No. WO2018/167038 (Vorobjov et al.). Typical methods include extracting cannabinoids from Cannabis plant material using an extractor with super critical, critical or near-critical fluids such as carbon dioxide, nitrous oxide, ethylene, ethane, propane or chlorodifluoromethane. In some embodiments, supercritical carbon dioxide is used, which behaves like an organic solvent with the solubilization characteristics of a liquid and the permeabilization characteristics of a gas.

As described herein, different cannabinoids are known to have different pharmaceutical effects. Thus, in certain situations, it may be desirable to provide a formulation including only one cannabinoid, such as THC or CBD. In one or more embodiments, the formulations described herein include CBD and do not include a substantial amount of THC. In one or more embodiments, the formulations described herein include THC and do not include a substantial amount of CBD.

In certain other situations, it may be desirable to provide a formulation including more than one cannabinoid, such as THC and CBD. In one or more embodiments, the formulations described herein include both THC and CBD. It is understood that the ratio of THC to CBD in any particular formulation may be varied to balance the psychotropic and therapeutic effects of the formulation. In embodiments wherein the formulation includes more than one cannabinoid, such as THC and CBD, the ratio between cannabinoids may be controlled. The ratio of THC to CBD may be, for example, at least 1:1 by weight, at least 1:2 by weight, at least 1:3 by weight, at least 1:4 by weight, at least 1:5 by weight, at least 1:6 by weight, at least 1:7 by weight, at least 1:8 by weight, at least 1:10 by weight, at least 1:12 by weight, at least 1:14 by weight, at least 1:16 by weight, at least 1:18 by weight, or at least 1:20 by weight. The ratio of THC to CBD may be, for example, at most 20:1 by weight, at most 18:1 by weight, at most 16:1 by weight, at most 14:1 by weight, at most 12:1 by weight, at most 10:1 by weight, at most 8:1 by weight, at most 7:1 by weight, at most 6:1 by weight, at most 5:1 by weight, at most 4:1 by weight, at most 3:1 by weight, or at most 1:1 by weight. The ratio of THC to CBD may be, for example, from 1:20 to 20:1, such as from 1:8 to 8:1, such as from 1:4 to 4:1, such as 1:1. In one or more other embodiments, the ratio of THC to CBD in a formulation that includes both THC and CBD may be expressed as a molar ratio.

The cannabinoid may be provided in any form suitable for formulation as a solution. In certain embodiments, the cannabinoid may be provided as a solid, such as a powder or a micronized powder, a resin, a semi-solid, or as a liquid, such as a stock solution. Any suitable form of cannabinoid compatible with preparation of a solution may be used for the formulations of the present disclosure. The cannabinoid is dissolved in the formulations described herein to form a solution.

Pharmaceutical grade cannabidiol is typically a white, crystalline powder, stored in room temperature in sealed aluminum bags to limit exposure to air and light. Pharmaceutical grade delta 9 tetrahydrocannabinol is a transparent, amber viscous liquid, typically stored at a temperature of −20° C. or at a temperature of 2° C. to 8° C. The delta 9 tetrahydrocannabinol is typically stored with argon atmosphere, in sealed amber glass vials, having limited exposure to light.

The formulations of the present disclosure are solutions (i.e., solution formulations or solution compositions). That is, the formulations include one or more cannabinoids dissolved in the formulations (i.e., solubilized in the propellant, cosolvent, and optional other components) to form solutions. Herein, a “solution” is a homogeneous solution that does not have particulate material visible to the unaided human eye.

The stability of a solution across a range of temperatures may be a factor to consider when selecting a formulation for a metered dose inhaler. In one or more embodiments, the components of solution formulations described herein may remain dissolved at a temperature of at least 25° C., at least 20° C., at least 15° C., at least 10° C., at least 9° C., at least 8° C., at least 7° C., at least 6° C., at least 5° C., at least 4° C., at least 3° C., at least 2° C., at least 1° C., or at least 0° C.

Solution and suspension formulations are fundamentally different metered dose inhaler formulation approaches. Different factors need to be considered when undertaking the development of products using either of these formulation approaches. Accordingly, it is not possible to apply the same knowledge and understanding of suspension formulations to solution formulations. In solutions, solubility of the active pharmaceutical ingredient (API) in the propellant, and optional cosolvent, is a key consideration. Various strategies can be used to improve solubility via use of additional excipients such as polyethylene glycol or water. Typically, solutions give smaller aerosol particle size distributions than suspensions and are generally more efficient than suspensions, but the overall dose may be limited due to the amount of API that can be solubilized. The use of cosolvents in metered dose inhalers including solution formulations may influence droplet evaporation rates, and can also lead to changes in the resulting solid-state particles formed in the lung, which may impact the pharmacological uptake of the API compared with deposited API from a suspension. Also, some APIs are at higher risk of chemical degradation in solution formulations and often require specific formulation strategies, such as the use of stabilizing acids and specific selection of container closure systems to maximize chemical stability. These problems are specific to solutions and any teachings specific to suspensions do not necessarily overcome them.

The primary propellant of compositions (i.e., formulations) according to the disclosure is HFA-152a, also known as HFC-152a, R-152a, 1,1-difluoroethane, or DFE.

In one or more embodiments, the amount of HFA-152a by weight in the formulation is at least 50%, greater than 50%, at least 60%, greater than 60%, at least 70%, greater than 70%, at least 80%, greater than 80%, at least 85%, greater than 85%, at least 90%, or greater than 90%.

In one or more embodiments, the amount of HFA-152a by weight is between 50% and 99%, between 55% and 95%, between 60% and 90%, or between 70% and 80%. In one or more embodiments, HFA-152a is the sole propellant in the formulation. That is, the pharmaceutical product performance parameters, such as emitted dose and emitted particle size distribution, are not significantly different than if HFA-152a were the sole propellant in the formulation.

The propellant HFA-152a is very different from other propellants, including propellants HFA-227, HFA-134a, and the low GWP propellant HFO-1234ze(E). These propellants have different physical, chemical, and thermodynamic properties such as boiling point, vapor pressure, water solubility, liquid density, and surface tension. The differences in these properties make replacing one propellant with another, such as HFA-152a, without significantly compromising or altering pMDI product performance difficult to achieve. For example, the thermodynamic differences in propellant boiling point and vapor pressure can significantly affect pMDI aerosolization efficiency and give rise to differences in primary and secondary atomization mechanisms. Differences in dipole moment and polarity between the propellants can affect the solubility of APIs and excipients in the formulation. Differences in hygroscopicity between the propellants can affect moisture uptake, which could be problematic for solution formulations, particularly if physical stability due to moisture uptake or chemical degradation in which water is involved is likely. Chemical interactions of the different propellants with APIs and excipients may also be significantly different, which could affect the long-term chemical stability of the product over the intended shelf life. Different propellants interact chemically and physically with valve plastics and elastomeric components, which could give rise to differences in the types and amounts of extractables and leachables, as well as impact mechanical valve function. The thermodynamic properties of the propellants can give rise to different droplet particle sizes due to different evaporation rates and can also result in differences in spray characteristics such as spray force, temperature, and spray duration. Historically, the transition from CFC to HFA propellants has required significant efforts to develop new approaches to reformulate and develop capable hardware to achieve appropriate pMDI product performance. That is, it was not possible to simply directly substitute one propellant for another. Changing from a propellant such as HFO-1234ze(E), HFA-227, or HFA-134a to HFA-152a in a pMDI is equally challenging due to many of the factors highlighted above.

The total amount of formulation is desirably selected so that at least a portion of the propellant in the canister is present as a liquid after a predetermined number of medicinal doses have been delivered. The predetermined number of doses may be 5 doses to 300 doses, 30 doses to 200 doses, 60 doses to 200 doses, 60 doses to 120 doses, 60 doses, 120 doses, 200 doses, or any other number of doses. The total amount of formulation in the canister may be from 1.0 grams (g) to 30.0 g, 2.0 g to 20.0 g, or 5.0 g to 15.0 g. The total amount of formulation is typically selected to be greater than the product of the predetermined number of doses and the metering volume of the metering valve. In one or more embodiments, the total amount of formulation is greater than 1.1 times, greater than 1.2 times, greater than 1.3 times, greater than 1.4 times, or greater than 1.5 times the product of the predetermined number of doses and the metering volume of the metering valve. This typically ensures that the amount of each dose remains relatively constant through the life of the inhaler.

In one or more embodiments, the formulation may include one or more additional APIs. Cannabinoids are effective in the treatment of many conditions, and the efficacy of treatment may be improved when cannabinoids are co-administered with an additional API. Exemplary APIs can include those for the treatment of respiratory disorders, e.g., a bronchodilator, such as a short- or long-acting beta agonist, an anti-inflammatory (e.g., a corticosteroid), an anti-allergic, an anti-asthmatic, an antihistamine, a TYK inhibitor, an anesthetic, or an anticholinergic agent. Exemplary APIs can include terbutaline, ipratropium, oxitropium, tiotropium, beclomethasone, flunisolide, ciclesonide, cromolyn sodium, nedocromil sodium, ketotifen, azelastine, ergotamine, cyclosporine, aclidinium, umeclidinium, glycopyrronium (i.e., glycopyrrolate), salmeterol, formoterol, procaterol, indacaterol, carmoterol, milveterol, olodaterol, vilanterol, abediterol, omalizumab, zileuton, insulin, pentamidine, calcitonin, leuprolide, alpha-I-antitrypsin, interferon, triamcinolone, nintedanib, lidocaine, a pharmaceutically acceptable salt or ester of any of the listed APIs, or a mixture of any of the listed APIs, their pharmaceutically acceptable salts or their pharmaceutically acceptable esters.

In all embodiments wherein the formulation includes one or more additional APIs, the API(s) are dissolved in the formulation (i.e., as a solution). In the event that a combination of two or more APIs are used, all of the APIs are in solution.

The amount of cannabinoid delivered may be determined by the required dose per actuation and the pMDI metering valve size, that is, the size of the metering chamber, which may be between 5 microliters (μL or mcl) and 200 microliters, between 25 microliters and 200 microliters, between 25 microliters and 150 microliters, between 25 microliters and 100 microliters, between 50 microliters and 100 microliters, between 25 microliters and 65 microliters, between 50 microliters and 65 microliters, or between 50 microliters and 63 microliters.

As described herein, the dose delivered by a metered dose inhaler may be expressed as the amount of cannabinoid that exits the actuator with each actuation (also referred to herein as the “ex-actuator” dose). This amount refers to the amount of cannabinoid that exits the nozzle, which may be substantially similar to the amount of cannabinoid delivered to a subject. The dose delivered by a metered dose inhaler may additionally or alternately be expressed as the amount of cannabinoid that exits the inhaler valve with each actuation. As described in the Examples of the present disclosure, the amount of API that exits the inhaler valve and the amount of API that exits the nozzle may or may not be substantially similar.

In certain embodiments, typical formulations of the present disclosure include the cannabinoid in an amount of at least 0.05 milligram per actuation (mg/actuation), or at least 0.5 mg/actuation. In certain embodiments, formulations of the present disclosure include the cannabinoid in an amount of less than 6.0 mg/actuation.

In one or more embodiments, typical formulations of the present disclosure include the cannabinoid in an amount of at least 0.05 mg/actuation, at least 0.075 mg/actuation, at least 0.1 mg/actuation, at least 0.2 mg/actuation, at least 0.3 mg/actuation, at least 0.4 mg/actuation, at least 0.5 mg/actuation, at least 0.75 mg/actuation, at least 1.0 mg/actuation, at least 1.5 mg/actuation, at least 2.0 mg/actuation, at least 3.0 mg/actuation, or at least 4.0 mg/actuation. In embodiments, typical formulations of the present disclosure include the cannabinoid in an amount of less than 6.0 mg/actuation, at most 5.0 mg/actuation, at most 4.0 mg/actuation, at most 3.0 mg/actuation, at most 2.0 mg/actuation, at most 1.5 mg/actuation, or at most 1.0 mg/actuation. In some preferred embodiments, formulations of the present disclosure include the cannabinoid in an amount of 0.05 mg/actuation to 6.0 mg/actuation, such as 0.1 mg/actuation to 5.0 mg/actuation, 0.5 mg/actuation to 1.5 mg/actuation, or 1.0 mg/actuation to 3.0 mg/actuation.

The amount of cannabinoid delivered by a metered dose inhaler may depend on the composition of cannabinoid(s) in the formulation. For example, a formulation including only CBD may include an amount of CBD such that from 0.05 mg/actuation to 6.0 mg/actuation, such as from 0.5 mg/actuation to 3.0 mg/actuation, or from 0.5 mg/actuation to 1.5 mg/actuation is delivered. A formulation including only THC may include an amount of THC such that from 0.2 mg/actuation to 6.0 mg/actuation, such as from 0.5 mg/actuation to 3.0 mg/actuation, such as 0.5 mg/actuation to 1.5 mg/actuation is delivered. A formulation including both THC and CBD may include an amount of THC and CBD such that 0.05 mg/actuation to 6.0 mg/actuation of each of THC and CBD is delivered. In some embodiments, a total of 0.05 mg/actuation to 6.0 mg/actuation of combined THC and CBD is delivered.

It should be noted that the amount of cannabinoid delivered by a metered dose inhaler is the result of a specific combination of multiple elements, including the concentration of cannabinoid in the formulation, the size of the actuator used, and the volume of the valve. Each of these metrics must be carefully considered when selecting an amount of cannabinoid to be delivered with each metered dose inhaler actuation.

In one or more embodiments, the concentration of cannabinoid may be described by the weight percentage of cannabinoid in the complete formulation. The formulation may include at least 0.5 mg/mL, at least 1.0 mg/mL, at least 1.5 mg/mL, at least 2.0 mg/mL, at least 2.5 mg/mL, at least 3.0 mg/mL, at least 3.5 mg/mL, at least 4.0 mg/mL, at least 5.0 mg/mL, at least 6.0 mg/mL, at least 7.0 mg/mL, at least 8.0 mg/mL, at least 9.0 mg/mL, at least 10 mg/mL, at least 12 mg/mL, at least 14 mg/mL, at least 15 mg/mL, at least 16 mg/mL, at least 17 mg/mL, at least 18 mg/mL, at least 19 mg/mL, at least 20 mg/mL, at least 25 mg/mL, at least 30 mg/mL, at least 35 mg/mL, at least 40 mg/mL, at least 45 mg/mL, at least 50 mg/mL, at least 55 mg/mL, at least 60 mg/mL, at least 65 mg/mL, at least 70 mg/mL, at least 75 mg/mL, at least 80 mg/mL, at least 85 mg/mL, at least 90 mg/mL, at least 95 mg/mL, at least 100 mg/mL, at least 110 mg/mL, at least 120 mg/mL, at least 130 mg/mL, at least 140 mg/mL, at least 150 mg/mL, at least 160 mg/mL, at least 170 mg/mL, at least 180 mg/mL, at least 190 mg/mL, or at least 200 mg/mL of cannabinoid, such as CBD and/or THC.

The formulation may include at most 240 mg/mL, at most 230 mg/mL, at most 220 mg/mL, at most 210 mg/mL, at most 200 mg/mL, at most 190 mg/mL, at most 180 mg/mL, at most 160 mg/mL, at most 140 mg/mL, at most 120 mg/mL, at most 100 mg/mL, at most 90 mg/mL, at most 80 mg/mL, at most 70 mg/mL, at most 60 mg/mL, at most 50 mg/mL, at most 40 mg/mL, or at most 30 mg/mL of cannabinoid, such as THC and/or CBD. The formulation may include, for example, from 0.5 mg/mL to 240 mg/mL, such as from 5 mg/mL to 80 mg/mL, from 10 mg/mL to 40 mg/mL, or about 16 mg/mL of cannabinoid, such as THC and/or CBD.

Typically, the formulation includes a cosolvent. One particularly useful cosolvent is ethanol. In one or more embodiments, ethanol is used as a cosolvent in solution formulations, i.e., where the cannabinoid is dissolved in the formulation. In one aspect, the cosolvent may aid in dissolving the cannabinoid whereas the cannabinoid may not be soluble in the formulation in the absence of a cosolvent.

Interestingly, it is reported herein that the amount of ethanol sufficient to solubilize a cannabinoid in a propellent differs depending on the propellant and cannabinoid(s) used in a given formulation. In particular, the amount of ethanol sufficient to solubilize THC, CBD, or a combination of THC and CBD in HFA-152a depends on the cannabinoid(s) in the formulation.

In one or more embodiments, the formulations described herein may include ethanol in an amount on a weight percent basis of the total formulation of at least 1%, at least 2%, at least 3%, at least 4%, at least 5%, at least 6%, at least 7%, at least 8%, at least 9%, at least 10%, at least 11%, at least 12%, at least 13%, at least 14%, or at least 15% of ethanol by weight. The formulations described herein generally may include ethanol in an amount on a weight percent basis of the total formulation of at most 20%, at most 25%, at most 14%, at most 13%, at most 12%, at most 11%, at most 10%, at most 9%, at most 8%, at most 7%, at most 6%, at most 5%, at most 4%, at most 3%, or at most 2%. The formulations described herein may include, for example, from 1% to 20% of ethanol, such as 1% to 15%, 1% to 8%, or 2% to 6% of ethanol by weight.

In one or more embodiments wherein a formulation includes CBD and THC, the formulation may include ethanol in an amount on a weight percentage basis of the total formulation from 1% to 15%, such as from 1% to 15%, or from 2% to 6%.

In one or more embodiments wherein a formulation includes CBD and does not include a substantial amount of THC, the formulation may include ethanol in an amount on a weight percentage basis of the total formulation from 1% to 15%, such as from 1% to 3%, or 1% to 2%.

In one or more embodiments wherein a formulation includes THC and does not include a substantial amount of CBD, the formulation may include ethanol in an amount on a weight percentage basis of the total formulation from 1% to 15%, such as from 1% to 4%.

In another aspect, a cosolvent may decrease deposition of the cannabinoid during actuation of the metered dose inhaler. For example, some propellants may evaporate rapidly as or before the formulation passes through the actuator. When ethanol is included in a formulation, it may delay evaporation of the formulation until it has passed through the actuator and out of the metered dose inhaler. In this way, inclusion of ethanol may decrease the amount of deposition of formulation on the components of the metered dose inhaler.

In one or more embodiments, the formulations described herein may include an amount of cosolvent sufficient to prevent occlusion of the actuator. In one or more embodiments, the formulations described herein may include an amount of cosolvent sufficient to prevent occlusion of the actuator after at least 5, at least 10, at least 20, at least 30, at least 60, at least 90, at least 120, at least 150, at least 180, or at least 240 actuations as compared to a formulation without a cosolvent.

In one or more embodiments, the formulation may include an amount of cosolvent to improve the consistency of delivered dose throughout the lifetime of the metered dose inhaler. For example, the formulation may include an amount of cosolvent sufficient to decrease deposition of the formulation during actuation of the metered dose inhaler. Deposition of the formulation during actuation of the metered dose inhaler may occlude the valve stem or upper valve stem component or otherwise clog the actuator nozzle. If the deposition of the formulation during actuation is decreased, the delivered dose consistency may stay relatively consistent throughout the lifetime of the metered dose inhaler. Deposition of the formulation during actuation may entirely prevent the metered dose inhaler from functioning. For example, deposition of the formulation may prevent further actuation of the metered dose inhaler. Deposition of the formulation may additionally or alternatively lower or otherwise undesirably alter the dose delivered by a metered dose inhaler.

In one or more embodiments, the metered dose inhalers described herein deliver an amount of cannabinoid per actuation that is consistent within a predetermined range throughout the lifetime of the metered dose inhaler. The metered dose inhalers described herein may deliver an amount of cannabinoid per actuation within 5%, within 10%, within 15%, within 20%, within 25%, within 30%, within 35%, or within 40% of an intended dose throughout the lifetime of the metered dose inhaler. As used herein, the “lifetime” and “unit life” of a metered dose inhaler encompasses the time taken to actuate a predetermined number of doses from the inhaler. “Lifetime” and “unit life” of a metered dose inhaler should be understood to be distinct from “shelf life” of a metered dose inhaler. While the “shelf life” of a metered dose inhaler typically refers to the length of time through which the product is considered stable and safe for delivery, the “unit life” or “lifetime” may be measured without a significant storage time, e.g., the unit may be tested through predetermined number of actuations in close series, such as over the course of one or several hours.

The consistency of dose delivered by a metered dose inhaler may additionally or alternatively be expressed as the minimum percentage of an initial intended dose delivered through the life of the metered dose inhaler. For example, a metered dose inhaler intended to deliver 1.0 mg/actuation which delivers between 1.0 and 0.8 mg/actuation throughout its lifetime may be said to have delivered at least 80% of an intended dose throughout its lifetime. In one or more embodiments, a metered dose inhaler may deliver at least 40%, at least 45%, at least 50%, at least 55%, at least 60%, at least 65%, at least 70%, at least 75%, or at least 80% of a predetermined dose per actuation throughout its lifetime.

In one or more embodiments, additional components (e.g., excipients) beyond propellant, cosolvent, and cannabinoid can be added to the formulation. These components may have various uses and functions, including, but not limited to, aiding in dissolution of the cannabinoid or other components, and/or aiding in chemical stabilization of cannabinoid or other components.

In one or more embodiments, the formulations described herein may also include additional components to confer a desired consumer property, such as scent, taste, color, or viscosity. Exemplary additional components include, for example, colorants or flavor or masking components.

Flavor or masking components may include flavonoids, terpenes or terpenoids. Non-limiting examples of flavonoids include quercetin, luteolin, vitexin, isovitexin, cannflavin A, cannflavin B, cannflavin C, apigenin, kaempferol, and orientin). Non-limiting examples of terpenoids include A3-Carene, B-Selinene, 3-pinene, f-phellandrene, beta-famesene, beta-caryophyllene, beta-pinene, beta-eudesmol, o-Terpinolene, alpha-pinene, o-phellandrene, o-Humulene, alpha-bergamotene, alpha-terpineol, alpha-terpinene, alpha-pinene, alpha-humulene, o-guaiene (t), a-Cedrene, alpha-bisabolol, Valencene (t), trans-Ocimene, trans-Ocimene, trans-Caryophyllene, Terpinolene, t=2-Pinanol (t), Selina-3,7-(11)-diene, Selina-3,7(11)-diene (t), Sabinene Hydrate, Nerol, Myrcene, Myrcene, Menthol, Linalool, Limonene, Isoborneol, Guaiol, Guaia-1(10),11-diene (t), Germacrene B (t), Geraniol, Farnesene (t), Eudesm-7(11)″en-4-ol (t), Elemene (t), cis-Ocimene, Caryophyllene oxide, Camphor, Camphene, Borneol and (+)Fenchol. Non-limiting examples of terpenes include Hemiterpenes, Monoterpenes, Sesquiterpenes, Diterpenes, Sesterterpenes, Triterpenes, Sesquiterpenes, Tetraterpenes, Polyterpenes, and Norisoprenoids as well as naturally present terpenes found in Cannabis plants, including but not limited to, myrcene, limonene, caryophyllene, pinene, terpinene, terpinolene, camphene, terpineol, phellandrene, carene, humulene, pulegone, sabinene, geraniol, linalool, fenchol, borneol, eucalyptol, and nerolidol.

In one or more embodiments, the formulations described herein display physical stability such that no particles are visible for at least 6 months, such as at least 12 months or at least 18 months, and often from 6 months to 36 months under typical storage conditions (e.g., refrigeration at a temperature of 2° C. to 8° C.). In certain embodiments, formulations of the present disclosure preferably display chemical stability such that no degradation products are formed for at least 6 months, such as at least 12 months or at least 18 months, and often from 6 months to 36 months under typical storage conditions (e.g., refrigeration at a temperature of 2° C. to 8° C.).

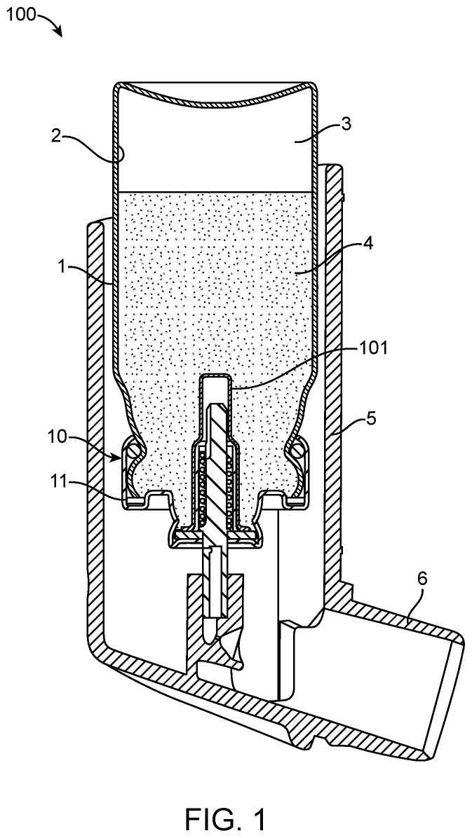

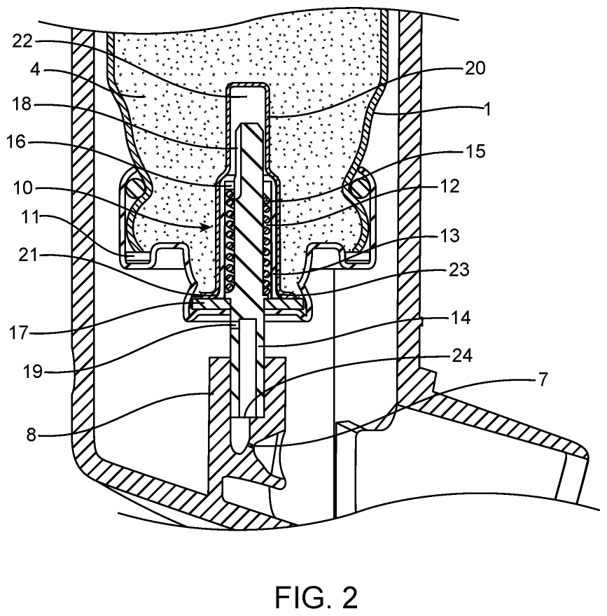

The various embodiments of formulations described herein can be utilized with any suitable inhaler. For example, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister 1 via a cap or ferrule 11 (typically made of aluminum or an aluminum alloy) which is generally provided as part of the valve assembly. Between the canister and the ferrule there may be one or more seals. In the embodiments shown in FIGS. 1 and 2 between the canister 1 and the ferrule 11 there are two seals including, e.g., an O-ring seal and a gasket seal. In one or more embodiments, the can may be uncoated, such as an uncoated aluminum canister. In one or more other embodiments, the can may be coated, such as with a silicon-containing coating. The coating may reduce the likelihood of deposition of the cannabinoid on the internal surface of the canister.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister 1 and outer walls 101 of the portion(s) of the metering valve 10 located within the canister define a formulation chamber 3 in which aerosol formulation 4 is contained.

The valve 10 shown in FIGS. 1 and 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol formulation 4 can pass from the formulation chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIGS. 1 and 2, allowing formulation to pass from the metering chamber 12 through a side hole 19 in the valve stem and through a stem outlet 24 to an actuator nozzle 7 then out to the patient. When the valve stem 14 is released, formulation enters into the valve 10, in particular into the pre-metering chamber 22, through the annular space 21 and thence from the pre-metering chamber through a groove 18 in the valve stem past the tank seal 16 into the metering chamber 12.

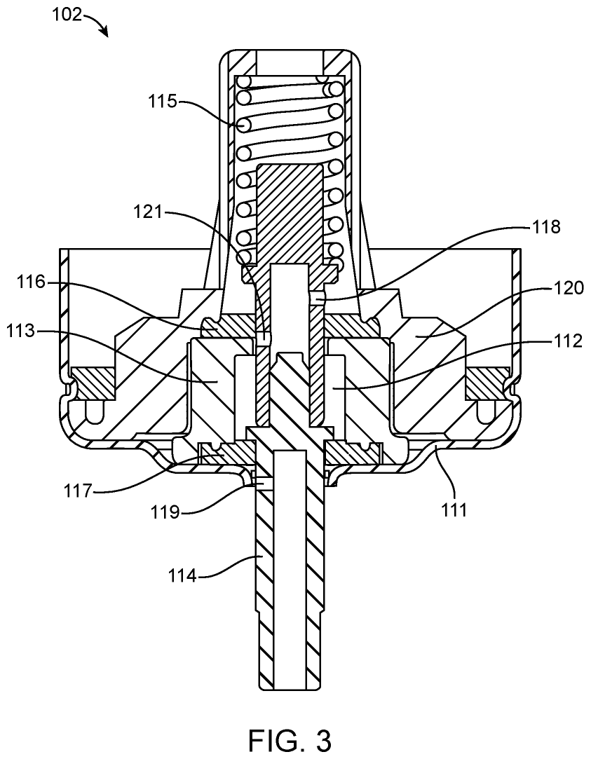

FIG. 3 shows another embodiment of a metered dose aerosol metering valve 102, different from the embodiment shown in FIGS. 1 and 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, formulation enters the metering chamber via orifices 121 and 118. The formulation's outward path from the metering chamber 112 when a dose is dispensed is via orifice 119.

Devices that may be used with medicament formulations of the present disclosure include those described in U.S. Pat. No. 6,032,836 (Hiscocks et al.), U.S. Pat. No. 9,010,329 (Hansen), and U.K. Patent GB 2544128 B (Friel).

The metered dose inhaler can include a dose counter for counting the number of doses. Suitable dose counters are known in the art, and are described in, for example, U.S. Pat. No. 8,740,014 (Purkins et al.); U.S. Pat. No. 8,479,732 (Stuart et al.); and U.S. Pat. No. 8,814,035 (Stuart), and U.S. Patent Application Publication No. 2012/0234317 (Stuart) all of which are incorporated by reference in their entirety with respect to their disclosures of dose counters.

Some coatings that can be used are described in U.S. Pat. No. 8,414,956 (Jinks et al.), U.S. Pat. No. 8,815,325 (David et al.), and United States Patent Application Publication No. 2012/0097159 (Iyer et al.), all of which are incorporated by reference in their entireties for their disclosure of coatings for inhalers and inhaler components. Suitable coatings may include polytetrafluoroethylene (PTFE), fluorinated ethylene propylene resins (FEP), and fluorocarbon polymer (FCP).

In one or more embodiments the actuator nozzle is sized so as to optimize the fine particle fraction (FPF) and/or respirable dose delivered of the formulation within the canister. In one or more embodiments the cross-sectional shape of the actuator nozzle is essentially circular or circular and has a predetermined diameter. In one or more embodiments where the cross-sectional shape of the actuator nozzle is non-circular, for example oval, an effective diameter may be determined by taking an average over the distances spanning the opening (e.g., the average of major and minor axes of an ellipse).

In one or more embodiments the exit orifice (effective diameter) of the actuator nozzle may be 0.08 mm or greater, 0.10 mm or greater, 0.12 mm or greater, 0.15 mm or greater, 0.175 mm or greater, 0.225 mm or greater, 0.3 mm or greater, or 0.4 mm or greater. In one or more embodiments, the exit orifice (effective diameter) of the actuator nozzle may be 0.5 mm or less, 0.4 mm or less, 0.3 mm or less, 0.225 mm or less, 0.175 mm or less, or 0.15 mm or less.

In one or more embodiments, the exit orifice (effective diameter) of the actuator nozzle may be 0.10 mm to 0.50 mm. In one or more embodiments, the exit orifice (effective diameter) of the actuator nozzle may be 0.15 mm to 0.50 mm. In one or more embodiments, the exit orifice (effective diameter) of the actuator nozzle may be 0.20 mm to 0.45 mm. In one or more embodiments, the exit orifice (effective diameter) of the actuator nozzle may be 0.25 mm to 0.40 mm. In one or more embodiments, the exit orifice (effective diameter) of the actuator nozzle may be 0.28 mm to 0.35 mm.

It should be appreciated by one of ordinary skill in the art that a given actuator nozzle exit orifice may not be suitable for delivery of any formulation, and that selection of a suitable actuator nozzle exit orifice for a given formulation involves considerable effort. Selection of a suitable actuator nozzle exit orifice may improve the consistency of dose delivered by a metered dose inhaler and/or decrease the likelihood of deposition of the formulation during actuation. An actuator nozzle exit orifice may additionally modify the properties of the aerosol delivered, such as fine particle fraction (FPF) and/or median mass aerodynamic diameter (MMAD) as described in more detail herein.

In one or more embodiments, the metered dose inhalers disclosed herein may deliver a dose comprising a particular fine particle fraction (FPF). As used herein, the FPF refers to the mass percentage of API particles with an aerodynamic diameter below 5 micrometers (μm) relative to the total emitted dose. FPF may be determined using any field standard method, such as using an impactor apparatus. As described herein, the FPF delivered by a metered dose inhaler is impacted by multiple considerations including actuator size and shape. In one or more embodiments, a metered dose inhaler may deliver an aerosol including a FPF of at least 5%, at least 10%, at least 15%, at least 20%, at least 25%, at least 30%, at least 35%, at least 40%, at least 45%, or at least 50%. In one or more embodiments, a metered dose inhaler may deliver an aerosol including a FPF of at most 90%, at most 85%, at most 80%, at most 75%, at most 70%, at most 65%, at most 60%, or at most 55%.

One metric used to describe a formulation delivered by a metered dose inhaler is the median mass aerodynamic diameter (MMAD). The MMAD is a measure of the size of particles delivered by a metered dose inhaler. Different MMAD measurements and ranges may be desirable in different applications. In one or more embodiments, a metered dose inhaler may deliver particles with a MMAD of at least 0.5 μm, at least 1 μm, at least 1.25 μm, at least 1.5 μm, at least 1.75 μm, at least 2 μm, at least 2.25 μm, at least 2.5 μm, at least 2.75 μm, at least 3 μm, at least 3.5 μm, or at least 4 μm. In one or more embodiments, a metered dose inhaler may deliver particles with a MMAD of at most 10 μm, at most 9 μm, at most 8 μm, at most 7 μm, at most 6.5 μm, at most 6.0 μm, at most 5.5 μm, or at most 5 μm.

The metering valve of a metered dose inhaler may have any suitable volume to deliver a dose of the formulation. Selection of a metering valve volume may impact the amount of formulation delivered. Therefore, in embodiments wherein the metered dose inhaler comprises a formulation comprising one or more cannabinoids, the metering valve volume will impact the amount of cannabinoid delivered. In one or more embodiments, the metering valve volume may be at least 30 μL, at least 40 μL, at least 50 μL, at least 60 μL, at least 70 μL, at least 80 μL, at least 90 μL, at least 100 μL, at least 110 μL, at least 120 μL, at least 130 μL, at least 140 μL, at least 150 μL, at least 175 μL, at least 200 μL, at least 225 μL, at least 250 μL, at least 275 μL, at least 300 μL, or at least 400 μL. The metering valve volume may be at most 500 μL, at most 450 μL, at most 400 μL, at most 350 μL, at most 300 μL, at most 275 μL, at most 250 μL, at most 225 μL, at most 200 μL, at most 175 μL, at most 150 μL, at most 125 μL, at most 100 μL, at most 75 μL, or at most 50 μL. The metering valve volume may be from 30 to 100 μL, such as 40 to 80 μL, or 50 to 70 μL.

It should be appreciated by one of ordinary skill in the art that a given valve volume may not be suitable for delivery of any formulation, and that selection of a valve volume for a given formulation involves considerable effort.

In one or more embodiments, the metered dose inhaler is manufactured by pressure filling. In pressure filling, the liquid or powdered medicament, combined with one or more excipients (e.g., co-solvents), is placed in a suitable aerosol container (i.e., canister) capable of withstanding the vapor pressure of the propellant and fitted with a metering valve prior to filling. The propellant is then forced as a liquid through the valve into the container. In an alternate process of pressure filling, the particulate API is combined in a process vessel with propellant and one or more excipients (e.g., cosolvents), and the resulting API solution is transferred through the metering valve fitted to a suitable metered dose inhaler container.

In one or more embodiments, the metered dose inhaler is manufactured by cold filling. In cold filling, the liquid or powdered medicament is combined with one or more excipients (e.g., co-solvents) and propellant that is chilled below its boiling point and, optionally, one or more excipients are added to the metered dose inhaler container. In addition, a metering valve is fitted to the container post-filling.

For both pressure filling and cold filling processes, additional steps, such as mixing, sonication, and homogenization of the formulation may be optionally employed.

### EMBODIMENTS

Embodiment 1 is a metered dose inhaler comprising: a metering valve; a canister; and an actuator comprising an actuator nozzle; wherein the canister comprises a formulation, the formulation comprising a propellant comprising HFA-152a, at least 1% of ethanol by weight, and one or more cannabinoids; and wherein the one or more cannabinoids is dissolved in the formulation to form a solution.

Embodiment 2 is the metered dose inhaler of embodiment 1, wherein the metered dose inhaler delivers 1.0 milligram (mg) per actuation to 3.0 mg/actuation of the one or more cannabinoids. Embodiment 3 is the metered dose inhaler of embodiment 2, wherein the formulation comprises at most 15% of ethanol by weight. Embodiment 4 is the metered dose inhaler of embodiment 3, wherein the formulation comprises at most 8% ethanol by weight. Embodiment 5 is the metered dose inhaler of any one of embodiments 1 to 4, wherein the one or more cannabinoids comprises tetrahydrocannabinol (THC) and cannabidiol (CBD). Embodiment 6 is the metered dose inhaler of embodiment 5, wherein the ratio of THC to CBD is 1:1 by weight.

Embodiment 7 is a metered dose inhaler comprising: a metering valve; a canister; and an actuator comprising an actuator nozzle; wherein the canister comprises a formulation, the formulation comprising a propellant comprising HFA-152a, at least 1% of ethanol by weight, and THC; wherein the THC is dissolved in the formulation to form a solution.

Embodiment 8 is the metered dose inhaler of embodiment 7, wherein the formulation comprises at most 15% of ethanol by weight. Embodiment 9 is the metered dose inhaler of embodiment 8, wherein the formulation comprises at most 4% ethanol by weight. Embodiment 10 is the metered dose inhaler of any one of embodiments 5 to 9, wherein the THC is delta-9-tetrahydrocannabinol. Embodiment 11 is the metered dose inhaler of any one of embodiments 7 to 10, wherein the metered dose inhaler delivers 0.5 mg/actuation to 1.5 mg/actuation of THC.

Embodiment 12 is a metered dose inhaler comprising: a metering valve; a canister; and an actuator comprising an actuator nozzle; wherein the canister comprises a formulation, the formulation comprising a propellant comprising HFA-152a, at least 1% of ethanol by weight, and CBD; wherein the CBD is dissolved in the formulation to form a solution. Embodiment 13 is the metered dose inhaler of embodiment 12, wherein the formulation comprises at most 15% of ethanol by weight. Embodiment 14 is the metered dose inhaler of embodiment 13, wherein the formulation comprises at most 3% of ethanol by weight.

Embodiment 15 is the metered dose inhaler of any one of embodiments 12 to 14, wherein the metered dose inhaler delivers 0.5 mg/actuation to 1.5 mg/actuation of CBD.

Embodiment 16 is the metered dose inhaler of any preceding embodiment, further comprising an excipient. Embodiment 17 is the metered dose inhaler of any preceding embodiment, wherein HFA-152a is the sole propellant. Embodiment 18 is the metered dose inhaler of any preceding embodiment, wherein the formulation comprises an amount of ethanol sufficient to decrease deposition of the formulation within the valve or actuator during actuation of the metered dose inhaler as compared to a formulation free of ethanol. Embodiment 19 is the metered dose inhaler of any preceding embodiment, wherein the formulation comprises an amount of ethanol sufficient to decrease deposition of the formulation within the valve or actuator during actuation of the metered dose inhaler as compared to a formulation free of excipient after at least 15 actuations. Embodiment 20 is the metered dose inhaler of any preceding embodiment, wherein the metered dose inhaler delivers a consistent dose of cannabinoid through at least 60 actuations. Embodiment 21 is the metered dose inhaler of any preceding embodiment, wherein the metered dose inhaler demonstrates a through-unit life dose consistency of at least 0.5 mg/actuation. Embodiment 22 is the metered dose inhaler of any preceding embodiment, wherein the metered dose inhaler demonstrates a through-life dose consistency of at least 1.0 mg/actuation. Embodiment 23 is the metered dose inhaler of any preceding embodiment, wherein the metered dose inhaler delivers at least 50% of a predetermined dose per actuation through its lifetime. Embodiment 24 is the metered dose inhaler of any preceding embodiment, wherein the metered dose inhaler delivers particles with a mean mass aerodynamic diameter of 2.0 μm to 4.0 μm. Embodiment 25 is the metered dose inhaler of any preceding embodiment, wherein the metered dose inhaler delivers an aerosol including a fine particle fraction of 10% to 70%. Embodiment 26 is the metered dose inhaler of any preceding embodiment, wherein the metered dose inhaler delivers particles with a median mass aerodynamic diameter of 1.5 μm to 6 μm. Embodiment 27 is the metered dose inhaler of any preceding embodiment, wherein the metered dose inhaler delivers an amount of cannabinoid per actuation within 35% of an intended dose throughout the lifetime of the metered dose inhaler.

### EXAMPLES

### Example 1

In this Example, the visual solubility of formulations including Δ-9-THC, CBD, or a mixture of Δ-9-THC and CBD in HFA-152a with different amounts of ethanol were assessed.

Saturated solutions including HFA-152a, ethanol, and 15.87 mg/mL CBD were prepared according to TABLE 1. Formulations including HFA-152a and 15.87 mg/mL of Δ-9-THC were prepared according to TABLE 1. Formulations including HFA-152a, ethanol, 15.78 mg/mL of CBD, and 15.78 mg/mL of Δ-9-THC were prepared according to TABLE 1. Each formulation was prepared in ambient conditions. An aliquot of each formulation was stored for 14 days, either in ambient conditions or refrigerated at 5° C. After 14 days, visual solubility of each formulation was measured. A cloudy solution or evidence of particulate, precipitation, or residues indicated that the CBD and/or Δ-9-THC was not visually soluble. The results of this analysis are summarized in TABLE 1. It was also observed that all formulations including THC were slightly yellow.

TABLE 1
Results of visual solubility of Δ-9-THC and/or CBD
in HFA-152a with different amounts of ethanol. Crossed
out boxes indicate that the combination was not tested.

| | CBD | Δ-9-THC | Δ-9-THC + CBD |
|---|---|---|---|
| 1% ethanol by weight | Soluble | | |
| 2% ethanol by weight | soluble | | |
| 3% ethanol by weight | soluble | | |
| 4% ethanol by weight | | Soluble | soluble |
| 5% ethanol by weight | | Soluble | |
| 6% ethanol by weight | | Soluble | Soluble |
| 8% ethanol by weight | | | Soluble |

The solubility or insolubility of each formulation was observed to be the same whether the formulation was stored at 5° C. or in ambient conditions. From this Example, it was learned that CBD was soluble at a concentration of at least 15.87 mg/mL in formulations including HFA-152a and 1-3% ethanol by weight. It was learned that Δ-9-THC was soluble at a concentration of at least 15.87 mg/mL in formulations including HFA-152a and 4%, 5%, or 6% ethanol by weight. It was also learned that the mixture of at least 15.87 mg/mL of Δ-9-THC and at least 15.87 mg/mL of CBD was soluble in formulations including HFO-1234ze(E) and 4%, 6%, or 8% ethanol by weight.

### Example 2

In this Example, the equilibrium saturated solubility of Δ-9-THC and CBD individually in HFA-152a. was measured.

Formulations of either CBD or Δ-9-THC were prepared in HFA-152a without any additional cosolvents. Saturated solutions were prepared by adding an excess of drug to the propellant to ensure saturated solubility was achieved. Each solution was covered with aluminum foil to prevent UV light from reaching the drug. A first set of solutions was then shaken at room temperature for 4 days. A second set of solutions was stored at 5° C. and manually shaken several times per day. After 4 days, each solution was filtered to remove undissolved CBD and/or Δ-9-THC, and the concentration of each cannabinoid was measured. Formulations were assessed for solubility at room temperature and at 5° C.

From this Example, it was learned that the equilibrium saturated solubility of CBD in HFA-152a was at least 15.7 mg/mL at room temperature and 16.4 mg/mL at 5° C. It was also learned that the equilibrium saturated solubility of Δ-9-THC in HFA-152a was at least 24.7 mg/mL at room temperature and 21.8 mg/mL at 5° C.

### Comparative Example 3

In this Example, the equilibrium saturated solubility of CBD or Δ-9-THC individually in HFA-227 and HFA-134a was measured.

Formulations of either CBD or Δ-9-THC were prepared in HFA-227 or HFA-134A. Saturated solutions were prepared by adding an excess of drug to the propellant to ensure saturated solubility was achieved. Each solution was shaken at room temperature for 4 days, after which each solution was filtered to remove undissolved CBD and/or Δ-9-THC, and the concentration of each cannabinoid was measured. The equilibrium saturated solubility of each cannabinoid in each propellant was measured at room temperature.

It was observed that the equilibrium saturated solubility of CBD in HFA-134a was 2.5 mg/mL, and the equilibrium saturated solubility of CBD in HFA-227 was 1.8 mg/mL. It was also observed that the equilibrium saturated solubility of Δ-9-THC in HFA-134a was 2.3 mg/mL, and the equilibrium saturated solubility of THC in HFA-227 was 1.5 mg/mL.

From this Example, and from comparing the results of this Example to Example 2, it was learned that the solubility of CBD or THC in one propellant did not inform the solubility of the same molecule in a different propellant. For example, Δ-9-THC was observed to be more soluble than CBD in HFA-152a, but CBD was observed to be more soluble than Δ-9-THC in both HFA-134a and HFA-227. It was observed that both CBD and Δ-9-THC were more soluble in HFA-152a than either HFA-134a or HFA-227.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. All references and publications cited herein are expressly incorporated herein by reference in their entirety into this disclosure. Various features and aspects of the present disclosure are set forth in the following claims.

1. A metered dose inhaler comprising:
a metering valve;
a canister; and
an actuator comprising an actuator nozzle;
wherein the canister comprises a formulation, the formulation comprising a propellant comprising HFA-152a, at least 1% of ethanol by weight, and one or more cannabinoids; and
wherein the one or more cannabinoids is dissolved in the formulation to form a solution.

2. The metered dose inhaler of claim 1, wherein the metered dose inhaler delivers 1.0 milligram (mg) per actuation to 3.0 mg/actuation of the one or more cannabinoids.

3. The metered dose inhaler of claim 2, wherein the formulation comprises at most 15% of ethanol by weight.

4. (canceled)

5. The metered dose inhaler of claim 1, wherein the one or more cannabinoids comprises tetrahydrocannabinol (THC) and cannabidiol (CBD).

6. The metered dose inhaler of claim 5, wherein a ratio of THC to CBD is 1:1 by weight.

7. A metered dose inhaler comprising:
a metering valve;
a canister; and
an actuator comprising an actuator nozzle;
wherein the canister comprises a formulation, the formulation comprising a propellant comprising HFA-152a, at least 1% of ethanol by weight, and THC; and
wherein the THC is dissolved in the formulation to form a solution.

8. The metered dose inhaler of claim 7, wherein the formulation comprises at most 15% of ethanol by weight.

9. (canceled)

10. The metered dose inhaler of claim 7, wherein the THC is delta-9-tetrahydrocannabinol.

11. The metered dose inhaler of claim 7, wherein the metered dose inhaler delivers 0.5 mg/actuation to 1.5 mg/actuation of THC.

12. A metered dose inhaler comprising:
a metering valve;
a canister; and
an actuator comprising an actuator nozzle;
wherein the canister comprises a formulation, the formulation comprising a propellant comprising HFA-152a, at least 1% of ethanol by weight, and CBD; and
wherein the CBD is dissolved in the formulation to form a solution.

13. The metered dose inhaler of claim 12, wherein the formulation comprises at most 15% of ethanol by weight.

14. (canceled)

15. The metered dose inhaler of claim 12, wherein the metered dose inhaler delivers 0.5 mg/actuation to 1.5 mg/actuation of CBD.

16. The metered dose inhaler of claim 1, further comprising an excipient.

17. The metered dose inhaler of claim 1, wherein the propellant consists of HFA-152a.

18. (canceled)

19. The metered dose inhaler of claim 1, wherein the formulation comprises an amount of ethanol sufficient to decrease deposition of the formulation within the metering valve or actuator during actuation of the metered dose inhaler as compared to a formulation free of excipient after at least 15 actuations.

20. The metered dose inhaler of claim 1, wherein the metered dose inhaler delivers a consistent dose of cannabinoid through at least 60 actuations.

21. The metered dose inhaler of claim 1, wherein the metered dose inhaler demonstrates a through-unit life dose consistency of at least 0.5 mg/actuation.

22. (canceled)

23. The metered dose inhaler of claim 1, wherein the metered dose inhaler delivers at least 50% of a predetermined dose per actuation through its lifetime.

24-25. (canceled)

26. The metered dose inhaler of claim 1, wherein the metered dose inhaler delivers particles with a median mass aerodynamic diameter of 1.5 μm to 6 μm.

27. The metered dose inhaler of claim 1, wherein the metered dose inhaler delivers an amount of cannabinoid per actuation within 35% of an intended dose throughout a lifetime of the metered dose inhaler.
